(19) United States
(12) Patent Application Publication
(10) Pub. No.: US 2006/0076025 A1
(43) Pub. Date: Apr. 13, 2006

(54) Method of dieting

(76) Inventor: Kevin Kim, Los Angeles, CA (US)

Correspondence Address: PARK LAW FIRM, 3255 WILSHIRE BLVD, SUITE 1110, LOS ANGELES, CA, 90010, US

(21) Appl. No.: 10/960,338

(22) Filed: Oct. 7, 2004

(51) Int. Cl. A61B 19/00

(52) U.S. Cl. 128/921

(57) ABSTRACT

A method of dieting includes the steps of reducing the secretion of insulin in the metabolic system of body, reducing the carbohydrate from meals, reducing the size of meals, and reducing the sense of hunger and the craving for food. The step of reducing the secretion of insulin includes the step of having protein-intensified meals. The step of reducing the carbohydrate from meals includes the steps of having leafy vegetables and excluding fruits from meals. The step of reducing the size of meals includes the steps of breaking a given meal into multiple servings and having the servings with predetermined interval. The step of reducing the sense of hunger and the craving for food includes the steps of drinking enough water, excluding spicy foods from meals, and prohibiting exercise. In the final steps, the method of dieting further includes the steps of having balanced meals and excising regularly.

### BACKGROUND OF THE INVENTION

The invention is related to a method of dieting, which is very effective even in tough cases. The method of dieting starts with attacking the fundamental metabolic mechanism responsible for storing the fat in the body. Also, the method of dieting considers various aspects of difficulties of dieting such as coping with the hunger and the craving for food, accumulating stress of dieting, lingering dread of regaining the lost weight, etc. and provides an effective method to fight against them.

The conventional methods of dieting depend mainly on the brute force such as cutting down the food or driving a heavy schedule of physical exercise. The blind cutting down of food is so painful for the patients that it usually ends up with an outright failure. More than often, the heavy schedule of physical exercise is not effective especially when the patients are just not ready for that kind of hard work. In many cases, for the patients suffering from overweight, even a mild level of physical exercise is too much. On the top of it, it would be almost impossible to apply the two steps, cutting down the food and driving a heavy schedule of physical exercise, at the same time. Although the initial steps of the conventional method of dieting was successful in losing some weight for a short period with the blind diet and heavy exercise, it is totally different matter to keep out the lost weight for a reasonably long period. For the stress of blind dieting and heavy schedule of physical exercise is too much for the patient to stick to the original methods. The patients will break down sooner or later, and then the yo-yo dieting will be the unavoidable result, which is particularly dangerous and ineffective, because it leads to an immediate weight gain once the caloric restrictions are eased.

Accordingly, a need for an effective and safe method of dieting has been present all the time since the obesity became one of major threats to public health. This invention is directed to solve these important problems and satisfy the long-felt need.

### SUMMARY OF THE INVENTION

The present invention contrives to solve the disadvantages of the prior art.

An object of the invention is to provide a method of dieting, which is safe and effective.

Another object of the invention is to provide a method of dieting, which is good in diabetic control and helps people to learn how to eat right and how to control food intake.

Still other object of the invention is to provide a method of dieting, which help people to avoid the disease related to overweight such as heart disease, diabetes, arthritis, and hypertension.

The method of dieting includes the steps of reducing the secretion of insulin in the metabolic system of body, reducing the carbohydrate from meals, breaking a meal into multiple servings and having each serving with a predetermined interval, reducing the size of meals, and reducing the sense of hunger and the craving for food. The method of dieting further includes the step of maximizing the satiety with dietary meals, easing the stress of dieting with balanced meals, and encouraging the patient and keeping out the lost weight.

The step of reducing the secretion of insulin in metabolic system of body includes the steps of having meals without carbohydrate and eating the predetermined amount of food in each meal to increase the metabolism.

The step of reducing the carbohydrate from meals includes the steps of having protein-intensified meals, having vegetables with low carbohydrate, and having meals without fruits.

The step of reducing the sense of hunger and the craving for food includes the steps of drinking water as frequently as needed to quench the thirst, having meals without spicy foods, doing regular activities without exercise, and having meals without alcohol.

As a more specific embodiment, the method of dieting includes the serial steps of 1) reducing the carbohydrate from meals and reducing the sense of hunger and the craving for food, 2) reducing the carbohydrate from meals, reducing the sense of hunger and the craving for food, and maximizing the satiety with dietary meals, 3) reducing the carbohydrate from meals, reducing the sense of hunger and the craving for food, maximizing the satiety with dietary meals, and breaking a meal into two or more servings and having each serving with a predetermined intervals, 4) reducing the carbohydrate from reduced meals, reducing the sense of hunger and the craving for food, maximizing the satiety with dietary meals, reducing the size of meals, and having meals without vegetables and 5) eating balanced meal of reduced amount.

In this embodiment, the method of dieting further includes the steps of 6) repeating the above Steps 1 through 5 a predetermined times if necessary and 7) exercising regularly while eating balanced meals.

After people follow the steps 1 through 5, they get out from carbohydrate addiction, which enables the patients to maintain the reduced weight by balancing between daily intake of protein and carbohydrate. Body weight shall be maintained if the daily intake of protein is of equal amount as daily intake of carbohydrate. When the weight goes up due to taking too much carbohydrate, eating protein only meal 3 times a day as like following the step 1 without exercise for a week enables the patients to lower the craving and hunger and body weight shall be back to previous level. At that time, people need to eat balanced meal between carbohydrate and protein.

Each of the steps 1 through 5 lasts for at least 3 days up to 7 days. The last step, exercising regularly while eating predetermined amount of balanced meals, lasts for about two months. The embodiment also may include the step of restarting the method of dieting in the event of breaking the rule in the middle of the method. The Step 6 may be skipped if the desirable weight is reached already after Step 5.

Many other innovations, features, and advantages will be evident with the following description of the invention.

### BRIEF DESCRIPTION OF THE DRAWINGS

These and other features, aspects and advantages of the present invention will become better understood with reference to the accompanying drawings, wherein:

FIG. 1 is a flow chart of an embodiment of the method of dieting.

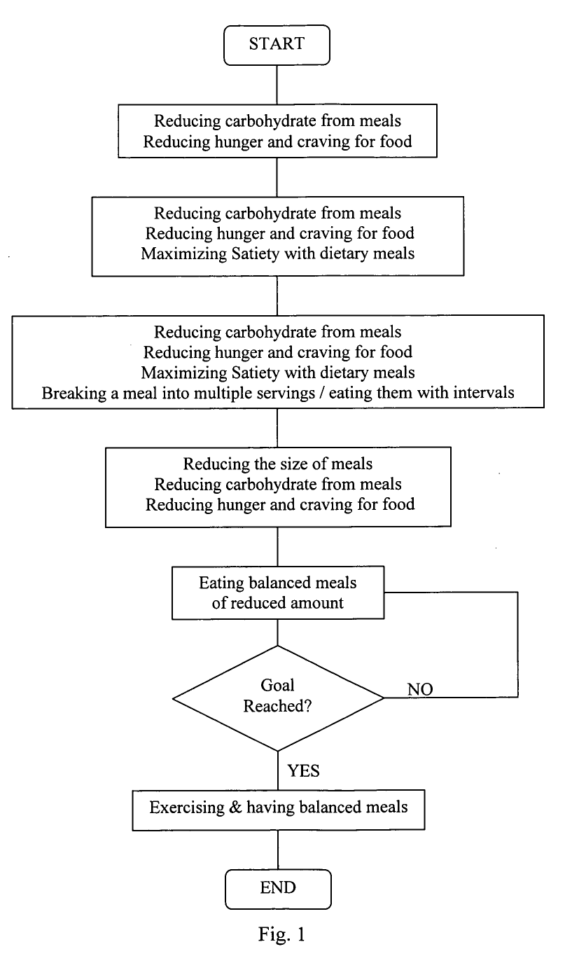

### DETAILED DESCRIPTION OF THE INVENTION

In wealthy industrialized countries, significant proportions of the population are obese these days, which is considered as a major threat to the public health. The modern society stricken by the reduction in physical activity and the high-calorie diets turns out to be a perfect place where the obesity prospers at the price of human health. On the top of that, many people may have a genetic propensity towards obesity, which was imprinted on the gene itself through a long history of harsh existence. Now, especially in a sedentary society, where high-energy food is available in abundant quantities in the context of decreased exercise, almost 30% of whole population are obese.

Even though the reason for the obesity is not clearly explained, one of the main reasons is a high insulin level in the metabolic system of the body. Insulin is a polypeptide hormone that regulates carbohydrate metabolism. That is, the insulin participates in the process of storing the fat in the body by forcing fat cells to take in glucose which is converted to fatty acids and by forcing adipose (fat) tissue to make fats from fatty acid esters. The lack of insulin causes the reverse.

Therefore, it is going to be a logical conclusion that if the insulin level is lowered in the body, storing fat will be also lowered.

Based on the results of scientific research in academia, this invention, Method of Dieting, suggests reducing the secretion of insulin in the metabolic system of body as a starting step.

To reduce the secretion of insulin in metabolic system of body, the patients need to have meals without carbohydrate and eat the same amount of food in each meal. Also, eating the predetermined and same amount of food in each meal helps to increase the metabolism itself.

In addition, the method of dieting includes the steps of reducing the carbohydrate from meals, reducing the size of meals, and reducing the sense of hunger and the craving for food. The method of dieting further includes the step of maximizing the satiety with dietary meals, easing the stress of dieting with balanced meals, and exercising to keep out the lost weight.

To reduce the carbohydrate from meals, the patients need to have protein-intensified meals to compensate for the reduced carbohydrate. And the vegetable with low carbohydrate, leafy vegetables for instance, is good for the diet. It is important to eat the leafy vegetables only with an amount less than the protein intake in mass. Since the fruit has large amount of carbohydrate, however, the patients should avoid fruits.

The step of breaking a meal into two or more servings and having each serving with a predetermined interval is a practice to reduce the absolute size of meals eventually. This is very important for dinner. The dinner is the heaviest meal for most of people. The overweight people can skip meals and then eat a huge meal, and are unable to stop meal before they feel full. Therefore, the patients need to practice not to eat up to the nines. Eating dinner twice with a reduced amount will be a practice before reducing the total amount of meals.

There are several ways to reduce the sense of hunger and the craving for food. Since our body does not distinguish hunger from thirst, the patients are recommended to drink water as frequently as needed to quench the thirst. And, the patients should avoid the spicy foods such as hot sauce, salsa, Tabasco sauce, etc. because they usually increase the appetite. For the same reason, alcohol is not allowed. Against the intuition, the patients should avoid physical exercise to curb the sense of hunger. It is true especially for the initial stages of the dieting. The initial starting weight of the patients is too heavy for the good level of exercise in many cases.

One way of maximizing the satiety with dietary meals is to chew about 20 times on each bite of protein food, which elongate the meal time. For, in order for the body to feel satiety, it takes about 5 to 10 minutes after eating. As it were, the fast eating results in overeating. Chewing more than 20 times gives power to control the amount of food. To make the patients more prepared, subconsciously, for extending the eating time, it is recommended to chew even the liquid foods such as water at least about 20 times. Then, the patients will be able to correct a bad habit of swallowing after one time or no chewing.

As a more specific embodiment, the method of dieting includes the serial steps of 1) reducing the carbohydrate from meals and reducing the sense of hunger and the craving for food, 2) reducing the carbohydrate from meals, reducing the sense of hunger and the craving for food, and maximizing the satiety with dietary meals, 3) reducing the carbohydrate from meals, reducing the sense of hunger and the craving for food, maximizing the satiety with dietary meals, and breaking a meal into multiple servings and having each serving with a predetermined intervals, 4) reducing the carbohydrate from reduced meals, reducing the sense of hunger and the craving for food, maximizing the satiety with dietary meals, reducing the size of meals, and having meals without vegetables, and 5) reducing the size of meals, reducing the carbohydrate from meals with protein reduced roughly by half, reducing the sense of hunger and the craving for food with water of increased amount, and eating balanced meal of reduced amount.

Step 5, eating balanced meals, is a resting step. Eating protein-intensified meals only for more than 2 weeks makes people stressful and tired of eating protein. The stress and fatigue increases the chance of giving up the diet plan. Therefore, the patients need a break for a while to relieve the stress from dieting.

In a balanced meal, the breakfast has to be easy and simple with a choice of carbohydrate. The craving for carbohydrate, which is highest in dinner time, can be relieved by eating protein. Eating balanced meals helps to keep out the lost weight.

A balanced meal includes a breakfast with the predetermined amount of carbohydrate with reduced calorie of daily meal FDA recommends, a lunch with half of predetermined amount of carbohydrate with reduced calorie of daily meal FDA recommends and half of predetermined amount of protein with reduced calorie of daily meal FDA recommends, and a dinner with predetermined amount of protein with reduced calorie of daily meal FDA recommends.

In this embodiment, the method of dieting further includes the steps of 6) repeating the above Steps 1 through 5 a predetermined times if necessary, 7) exercising regularly while eating balanced meals, and 8) repeating Steps 1 through 7.

People need to exercise physically when they feels light after losing more than 20 lbs. Exercise will not only encourage the patients, but also be the key to maintain the reduced weight.

Each of Steps 1 through 5 lasts for at least about 3 days up to about 7 days. For example, the patients need to stay away from carbohydrate for at least about 3 days in order to get out of the carbohydrate addiction.

Step 7, exercising regularly while eating predetermined amount of balanced meals, lasts for about two months. Exercise such as walking about 10,000 steps is recommended. Usually, people are not desperate to lose more weight after losing about 30 lbs. The achievement curve of the patients becomes a plateau, which is not responding to the treatment. After some break of this kind, the patients can repeat the whole method from the first step in order to lose another 30 lbs.

The embodiment also may include the step of restarting the method of dieting in the event of breaking the rule in the middle of the method. The Step 6 may be skipped if the desirable weight is reached already right after Step 5.

In the embodiments of the method of dieting, a food diary, as shown in Table 1, helps a lot. The critical dieting points can be checked at a glance using the food diary. The food diary has columns for date, food (protein, carbohydrate, vegetables), amount, hunger, water, spicy, eat fast, etc. for the three meals (breakfast, lunch, and dinner).

TABLE 1

| | | | | | | | | | | | | | | | | Eat | | | | | | | | | | |
|---|---|---|---|---|---|---|---|---|---|---|---|---|---|---|---|---|---|---|---|---|---|---|---|---|---|---|
| Date | | Food | | | Amount | | | Hunger | | | Water | | | Spicy | | Fast | | Food | | | Amount | | | Hunger | | |
| / | B | Protein | Carb | Veg. | ½ | 1 | 2 | 0 | 5 | 9 | 1 | 2 | 3 | Y | N | Y | N | Protein | Carb | Veg. | ½ | 1 | 2 | 0 | 5 | 9 |
| | L | Protein | Carb | Veg. | ½ | 1 | 2 | 0 | 5 | 9 | 1 | 2 | 3 | Y | N | Y | N | Protein | Carb | Veg. | ½ | 1 | 2 | 0 | 5 | 9 |
| | D | Protein | Carb | Veg. | ½ | 1 | 2 | 0 | 5 | 9 | 1 | 2 | 3 | Y | N | Y | N | Protein | Carb | Veg. | ½ | 1 | 2 | 0 | 5 | 9 |
| / | B | Protein | Carb | Veg. | ½ | 1 | 2 | 0 | 5 | 9 | 1 | 2 | 3 | Y | N | Y | N | Protein | Carb | Veg. | ½ | 1 | 2 | 0 | 5 | 9 |
| | L | Protein | Carb | Veg. | ½ | 1 | 2 | 0 | 5 | 9 | 1 | 2 | 3 | Y | N | Y | N | Protein | Carb | Veg. | ½ | 1 | 2 | 0 | 5 | 9 |
| | D | Protein | Carb | Veg. | ½ | 1 | 2 | 0 | 5 | 9 | 1 | 2 | 3 | Y | N | Y | N | Protein | Carb | Veg. | ½ | 1 | 2 | 0 | 5 | 9 |
| / | B | Protein | Carb | Veg. | ½ | 1 | 2 | 0 | 5 | 9 | 1 | 2 | 3 | Y | N | Y | N | Protein | Carb | Veg. | ½ | 1 | 2 | 0 | 5 | 9 |
| | L | Protein | Carb | Veg. | ½ | 1 | 2 | 0 | 5 | 9 | 1 | 2 | 3 | Y | N | Y | N | Protein | Carb | Veg. | ½ | 1 | 2 | 0 | 5 | 9 |
| | D | Protein | Carb | Veg. | ½ | 1 | 2 | 0 | 5 | 9 | 1 | 2 | 3 | Y | N | Y | N | Protein | Carb | Veg. | ½ | 1 | 2 | 0 | 5 | 9 |
| / | B | Protein | Carb | Veg. | ½ | 1 | 2 | 0 | 5 | 9 | 1 | 2 | 3 | Y | N | Y | N | Protein | Carb | Veg. | ½ | 1 | 2 | 0 | 5 | 9 |
| | L | Protein | Carb | Veg. | ½ | 1 | 2 | 0 | 5 | 9 | 1 | 2 | 3 | Y | N | Y | N | Protein | Carb | Veg. | ½ | 1 | 2 | 0 | 5 | 9 |
| | D | Protein | Carb | Veg. | ½ | 1 | 2 | 0 | 5 | 9 | 1 | 2 | 3 | Y | N | Y | N | Protein | Carb | Veg. | ½ | 1 | 2 | 0 | 5 | 9 |
| / | B | Protein | Carb | Veg. | ½ | 1 | 2 | 0 | 5 | 9 | 1 | 2 | 3 | Y | N | Y | N | Protein | Carb | Veg. | ½ | 1 | 2 | 0 | 5 | 9 |
| | L | Protein | Carb | Veg. | ½ | 1 | 2 | 0 | 5 | 9 | 1 | 2 | 3 | Y | N | Y | N | Protein | Carb | Veg. | ½ | 1 | 2 | 0 | 5 | 9 |
| | D | Protein | Carb | Veg. | ½ | 1 | 2 | 0 | 5 | 9 | 1 | 2 | 3 | Y | N | Y | N | Protein | Carb | Veg. | ½ | 1 | 2 | 0 | 5 | 9 |
| / | B | Protein | Carb | Veg. | ½ | 1 | 2 | 0 | 5 | 9 | 1 | 2 | 3 | Y | N | Y | N | Protein | Carb | Veg. | ½ | 1 | 2 | 0 | 5 | 9 |
| | L | Protein | Carb | Veg. | ½ | 1 | 2 | 0 | 5 | 9 | 1 | 2 | 3 | Y | N | Y | N | Protein | Carb | Veg. | ½ | 1 | 2 | 0 | 5 | 9 |
| | D | Protein | Carb | Veg. | ½ | 1 | 2 | 0 | 5 | 9 | 1 | 2 | 3 | Y | N | Y | N | Protein | Carb | Veg. | ½ | 1 | 2 | 0 | 5 | 9 |
| / | B | Protein | Carb | Veg. | ½ | 1 | 2 | 0 | 5 | 9 | 1 | 2 | 3 | Y | N | Y | N | Protein | Carb | Veg. | ½ | 1 | 2 | 0 | 5 | 9 |
| | L | Protein | Carb | Veg. | ½ | 1 | 2 | 0 | 5 | 9 | 1 | 2 | 3 | Y | N | Y | N | Protein | Carb | Veg. | ½ | 1 | 2 | 0 | 5 | 9 |
| | D | Protein | Carb | Veg. | ½ | 1 | 2 | 0 | 5 | 9 | 1 | 2 | 3 | Y | N | Y | N | Protein | Carb | Veg. | ½ | 1 | 2 | 0 | 5 | 9 |

Amount: Size of the fist
Hunger level before meal 0: not Hungry 9: starving
Water: cups
Eat fast: less than 10 chewing

The results of the method of dieting for ten patients of ages from twenties to fifties are shown in Table 2. In almost all the cases, there existed significant reduction in blood sugar level and insulin as well as the weight loss 5 over l to 2 months' period.

TABLE 2

| | P1 | P2 | P3 | P4 | P5 | P6 | P7 | P8 | P9 | P10 |
|---|---|---|---|---|---|---|---|---|---|---|
| gender | F | M | F | F | F | M | M | M | M | F |
| age | 53 | 29 | 20 | 56 | 44 | 52 | 39 | 40 | 33 | 23 |
| Weight to start | 180 | 221 | 163 | 197 | 131 | 328 | 216 | 142 | 264 lb | 310 lb |
| Weight to end | 157 | 179 | 138 | 179 | 116 | 310 | 208 | 125 | 221 lb | 223 lb |
| Blood sugar | N/A | N/A | N/A | 214 | N/A | 200 | 189 | N/A | N/A | N/A |
| before diet | | | | | | | | | | |
| Blood sugar | N/A | N/A | N/A | 104 | N/A | 169 | 120 | N/A | N/A | N/A |
| after diet | | | | | | | | | | |
| Insulin amount | N/A | N/A | N/A | N/A | N/A | 290 u | 36 u | N/A | N/A | N/A |
| before diet | | | | | | | | | | |
| Insulin amount | N/A | N/A | N/A | N/A | N/A | 0 | 0 | N/A | N/A | N/A |
| after diet | | | | | | | | | | |
| Starting date | May 2004 | May 2004 | June 2004 | July 2004 | May 2004 | July 2004 | June 2004 | May 2004 | June 2004 | September 2003 |
| Ending date | July 2004 | July 2004 | July 2004 | August 2004 | July 2004 | August 2004 | July 2004 | July 2004 | August 2004 | August 2004 |

While the invention has been shown and described with reference to different embodiments thereof, it will be appreciated by those skilled in the art that variations in form, detail, compositions and operation may be made without departing from the spirit and scope of the invention as defined by the accompanying claims.

What is claimed is:

1. A method of dieting comprising the steps of:
a) reducing the secretion of insulin in the metabolic system of body;
b) reducing the carbohydrate from meals;
c) breaking a meal into multiple servings and having each serving with a predetermined intervals;
d) reducing the size of meals; and
e) reducing the sense of hunger and the craving for food.

2. The method of claim 1, wherein the method further comprises the step of maximizing the satiety with dietary meals.

3. The method of claim 2, wherein the step of maximizing the satiety with dietary meals comprises the step of elongating the eating time to make a patient have enough time to feel the satiety.

4. The method of claim 3, wherein the step of elongating the eating time comprises the step of having food stay in a mouth for a predetermined period before swallowing.

5. The method of claim 4, wherein the step of having food stay in a mouth for a predetermined period before swallowing comprises the step of chewing food from about fifteen (10) to about thirty (30) times before swallowing.

6. The method of claim 5, wherein in the step of chewing food, food is chewed about twenty (20) times.

7. The method of claim 1, wherein the method further comprises the step of easing the stress of dieting.

8. The method of claim 7, wherein the step of easing the stress of dieting comprises the step of allowing a patient to have balanced meals for a predetermined period.

9. The method of claim 1, wherein the method further comprises the step of starting physical exercise on a regular basis.

10. The method of claim 1, wherein the step of reducing the secretion of insulin in metabolic system of body comprises the step of having meals without carbohydrate.

11. The method of claim 1, wherein the step of reducing the secretion of insulin in metabolic system of body comprises the step of eating same predetermined amount of food in each meal to increase the metabolism.

12. The method of claim 1, wherein the step of reducing the secretion of insulin in metabolic system of body comprises the steps of:
a) having meals without carbohydrate; and
b) eating same predetermined amount of food in each meal to increase the metabolism.

13. The method of claim 1, wherein the step of reducing the carbohydrate from meals comprises the step of having protein-intensified meals.

14. The method of claim 1, wherein the step of reducing the carbohydrate from meals comprises the step of having vegetables with low carbohydrate.

15. The method of claim 14, wherein the step of having vegetables with low carbohydrate comprises the step of having leafy vegetables with an amount less than the protein intake in mass.

16. The method of claim 1, wherein the step of reducing the carbohydrate from meals comprises the step of having meals without fruits.

17. The method of claim 1, wherein the step of reducing the carbohydrate from meals comprises the steps of:
a) having protein-intensified meals;
b) having vegetables with low carbohydrate; and
c) having meals without fruits.

18. The method of claim 1, wherein the step of reducing the size of meals comprises the step of reducing the size of meals based on the calorie of predetermined amount of daily meal FDA recommends.

19. The method of claim 1, wherein the step of breaking a meal into multiple servings and having each serving with a predetermined intervals comprises the step of breaking a dinner into about two (2) servings and having the servings about one (1) hour apart.

20. The method of claim 1, wherein the step of reducing the sense of hunger and the craving for food comprises the step of drinking water as frequently as needed to quench the thirst.

21. The method of claim 1, wherein the step of reducing the sense of hunger and the craving for food comprises the step of having meals without spicy foods.

22. The method of claim 1, wherein the step of reducing the sense of hunger and the craving for food comprises the step of doing regular activities without exercise.

23. The method of claim 1, wherein the step of reducing the sense of hunger and the craving for food comprises the step of having meals without alcohol.

24. The method of claim 1, wherein the step of reducing the sense of hunger and the craving for food comprises the steps of:
a) drinking water as frequently as needed to quench the thirst;
b) having meals without spicy foods;
c) doing regular activities without exercise; and
d) having meals without alcohol.

25. A method of dieting comprising the steps of:
a) reducing the carbohydrate from meals and reducing the sense of hunger and the craving for food;
b) reducing the carbohydrate from meals, reducing the sense of hunger and the craving for food, and maximizing the satiety with dietary meals;
c) reducing the carbohydrate from meals, reducing the sense of hunger and the craving for food, maximizing the satiety with dietary meals, and breaking a meal into multiple servings and having each serving with a predetermined intervals;
d) reducing the size of meals, reducing the carbohydrate from meals with protein reduced roughly by half, having meals without vegetable, and reducing the sense of hunger and the craving for food with water of increased amount; and
e) eating balanced meal of reduced amount.

26. The method of claim 25, wherein the method further comprises the step of repeating the steps a) through e).

27. The method of claim 25, wherein the method further comprises the step of exercising regularly and eating balanced meals.

28. The method of claim 27, wherein the step of exercising comprises the step of excising about 30 minutes at least every other day and eating balanced meal.

29. The method of claim 28, wherein the balanced meal comprises a breakfast with the predetermined amount of carbohydrate with reduced calorie of daily meal FDA recommends, a lunch with half of predetermined amount of carbohydrate with reduced calorie of daily meal FDA recommends and half of predetermined amount of protein with reduced calorie of daily meal FDA recommends, and a dinner with predetermined amount of protein with reduced calorie of daily meal FDA recommends.

30. The method of claim 25, wherein the step of exercising regularly and eating balanced meals lasts for about 2 months.

31. The method of claim 25, wherein the step of reducing the secretion of insulin in metabolic system of body comprises the step of having meals without carbohydrate.

32. The method of claim 25, wherein the step of reducing the secretion of insulin in metabolic system of body comprises the step of eating same predetermined amount of food in each meal to increase the metabolism.

33. The method of claim 25, wherein the step of reducing the secretion of insulin in metabolic system of body comprises the steps of:
a) having meals without carbohydrate; and
b) eating same predetermined amount of food in each meal to increase the metabolism.

34. The method of claim 25, wherein the step of reducing the carbohydrate from meals comprises the step of having protein-intensified meals.

35. The method of claim 34, wherein the step of having protein-intensified meals comprises eating protein-oriented meals about three times a day each with calorie of predetermined amount of daily meal FDA recommends.

36. The method of claim 25, wherein the step of reducing the carbohydrate from meals comprises the step of having vegetables with low carbohydrate.

37. The method of claim 36, wherein the step of having vegetables with low carbohydrate comprises the step of having leafy vegetables.

38. The method of claim 25, wherein the step of reducing the carbohydrate from meals comprises the step of having meals without fruits.

39. The method of claim 25, wherein the step of reducing the carbohydrate from meals comprises the steps of:
a) having protein-intensified meals;
b) having vegetables with low carbohydrate; and
c) having meals without fruits.

40. The method of claim 25, wherein the step of reducing the size of meals comprises the step of breaking a given meal into multiple servings.

41. The method of claim 25, wherein the step of reducing the size of meals comprises the step of having the servings one by one with predetermined intervals.

42. The method of claim 25, wherein the step of reducing the size of meals comprises the steps of:
a) breaking a given meal into multiple servings; and
b) having the servings one by one with predetermined intervals.

43. The method of claim 25, wherein the step of reducing the sense of hunger and the craving for food comprises the step of drinking water as frequently as needed to quench the thirst.

44. The method of claim 25, wherein the step of reducing the sense of hunger and the craving for food comprises the step of having meals without spicy foods.

45. The method of claim 25, wherein the step of reducing the sense of hunger and the craving for food comprises the step of doing activities without exercise.

46. The method of claim 25, wherein the step of reducing the sense of hunger and the craving for food comprises the step of having meals without alcohol.

47. The method of claim 25, wherein the step of reducing the sense of hunger and the craving for food comprises the steps of:
a) drinking water as frequently as needed to quench the thirst;
b) having meals without spicy foods;
c) doing activities without exercise; and
d) having meals without alcohol.

48. The method of claim 25, wherein the step of maximizing the satiety with dietary meals comprises the step of elongating the eating time to make a patient have enough time to feel the satiety.

49. The method of claim 48, wherein the step of elongating the eating time comprises the step of having food stay in a mouth for a predetermined period before swallowing.

50. The method of claim 49, wherein the step of having food stay in a mouth for a predetermined period before swallowing comprises the step of chewing food from about fifteen (10) to about thirty (30) times before swallowing.

51. The method of claim 50, wherein in the step of chewing food, food is chewed about twenty (20) times.

52. The method of claim 25, wherein the step of easing the stress of dieting comprises the step of allowing a patient to have balanced meals for a period.

53. The method of claim 52, wherein the balanced meal comprises a breakfast with the predetermined amount of carbohydrate with reduced calorie of daily meal FDA recommends, a lunch with half of predetermined amount of carbohydrate with reduced calorie of daily meal FDA recommends and half of predetermined amount of protein with reduced calorie of daily meal FDA recommends, and a dinner with predetermined amount of protein with reduced calorie of daily meal FDA recommends.

54. The method of claim 25, wherein each of the steps 1 through 5 lasts for at least about (3) days up to about seven (7) days.

55. The method of claim 54, wherein the predetermined period for each of the steps a) through e) is elongated until the instructions for the step are completed fully.

56. The method of claim 55, wherein the method of dieting comprises the steps of:
a) having protein-intensified meals with about two (2) fistful (600 g) of protein about three (3) times a day without carbohydrate to reduce the insulin level in the metabolism of body, having leafy vegetables only with an amount less than the protein intake in mass, having meals without spicy food, fruit, or alcohol to reduce the sense of hunger and the craving for food, doing regular activities without exercise, drinking about eight (8) cups of water (16 oz) a day, and eating more when feeling hungry, for at least about three (3) days up to about seven (7) days;
b) having protein-intensified meals with about two (2) fistful (600 g) of protein about three (3) times a day without carbohydrate to reduce the insulin level in the metabolism of body, chewing chewable food at least about twenty (20) times and keeping a liquid food in mouth for a predetermined period before swallowing, having meals without spicy food, fruit, or alcohol to reduce the sense of hunger and the craving for food, doing regular activities without exercise, and drinking about eight (8) cups of water (16 oz) a day, for at least about three (3) days up to about seven (7) days;
c) having protein-intensified meals with about two (2) fistful (600 g) of protein for breakfast and lunch without carbohydrate to reduce the insulin level in the metabolism of body, having dinner twice with about one (1) fistful (300 g) of protein only each with about one (1) hour interval, chewing chewable food at least about twenty (20) times and keeping a liquid food in mouth for a predetermined period before swallowing, having meals without spicy food, fruit, or alcohol to reduce the sense of hunger and the craving for food, doing regular activities without exercise, and drinking about eight (8) cups of water (16 oz) a day, for at least about three (3) days up to about seven (7) days;
d) having protein-intensified meals with about one (1) fistful (300 g) of protein about three (3) times a day without carbohydrate to reduce the insulin level in the metabolism of body, chewing chewable food at least about twenty (20) times and keeping a liquid food in mouth for a predetermined period before swallowing, having meals without spicy food, fruit, alcohol, or vegetable, doing regular activities without exercise, and drinking about twelve (12) cups of water (24 oz) a day, for at least about three (3) days up to about seven (7) days;
e) having balanced meals comprising a breakfast with about one (1) fistful (300 g) of carbohydrate, a lunch with about half fistful (150 g) of carbohydrate and about half fistful (150 g) of protein, and a dinner with about one (1) fistful (300 g) of protein, and chewing chewable food at least about twenty (20) times and keeping a liquid food in mouth for a predetermined period before swallowing, for at least about three (3) days up to about seven (7) days;
f) repeating the steps a) through e) one or more times; and
g) exercising about thirty (30) minutes every other day and having balanced meal as in the step e), for about two (2) months.

57. The method of claim 56, wherein the method of dieting further comprises the step of eating protein-only meals without exercise for about a week and then eating balanced meals, when the weight increases during step g).

58. The method of claim 25, wherein the method of dieting comprises repeating Steps a) through e) to lose more weight.

59. The method of claim 25, wherein the method of dieting further comprises the step of restarting the method of dieting in the event of breaking the rule in the middle of the method.
